(19) United States
(12) Patent Application Publication
(10) Pub. No.: US 2006/0105017 A1
(43) Pub. Date: May 18, 2006

(54) Methods for treatment of wounds using time release compositions

(75) Inventors: Xaverius F. Walboomers, Nijmegen (NL); Matthias Hoekstra, Amsterdam (NL); J. A. Jansen, Nijmegen (NL)

Correspondence Address: O'MELVENY & MYERS LLP, 610 NEWPORT CENTER DRIVE, 17TH FLOOR, NEWPORT BEACH, CA, 92660, US

(73) Assignee: GREYSTONE MEDICAL GROUP, INC., Memphis, TN (US)

(21) Appl. No.: 11/159,844

(22) Filed: Jun. 22, 2005

Related U.S. Application Data
(60) Provisional application No. 60/581,636, filed on Jun. 22, 2004.

(51) Int. Cl. A61K 6/083; B05D 3/00

(52) U.S. Cl. 424/423; 427/2.26

(57) ABSTRACT

A method for treatment of wounds associated with the insertion of a medical implant, wherein an inorganic therapeutic agent containing potassium, rubidium, calcium and zinc cations is applied to the wound site on a silicone or bioabsorbable membrane. The therapeutic composition is provided in a long lasting, timed delivery formulation to improve the efficacy of the therapeutic agent. The membrane may contain a micro-texture to further control delivery of the therapeutic agent to the wound site.

### RELATED APPLICATIONS

This application claims priority to U.S. Provisional Application Ser. No. 60/581,636, filed Jun. 22, 2004, entitled “Medical Applications Employing PHI-5 Loaded Silicone Membrane,” and is incorporated herein by reference as if fully set forth herein.

### FIELD OF THE INVENTION

This invention relates to the treatment of wounds, particularly wounds associated with medical implants which resist healing and thereby negatively interfere with the implant acceptance. It further relates to the use of timed release formulations of synthetic compositions containing the key ingredients of aqueous oak bark extract delivered on silicone or bioabsorbable membranes as an aid in the establishment and/or control over the chemical environment associated with extra cellular matrices.

### BACKGROUND OF THE INVENTION

Prior studies have shown that Oak Bark extract and synthetic compositions based on Oak Bark extract, described in U.S. Pat. Nos. 5,080,900 and 6,149,947, incorporated herein by reference, have a positive effect on the treatment of chronic wounds and skin disorders, including skin ulcers, particularly decubitus ulcers or bed sores. PHI-5 (Dermagenics, Memphis, Tenn., USA) is a novel material containing amongst others zinc and rubidium, which is based on analysis of red oak bark extract developed for treatment of chronic wounds.

Specifically, PHI-5 comprises a solution of potassium, rubidium, calcium and zinc cations. In one embodiment, PHI-5 comprises a pharmaceutically acceptable carrier, and an active ingredient of inorganic solids comprising 10-80 parts by weight of potassium ions, 0.00001-20 parts by weight of zinc ions, 0.01-10 parts by weight of calcium ions and rubidium ions in an amount of up to 40 parts by weight, all weights being based on the total weight of inorganic solids in their cationic form. Further description of PHI-5 is found in co-pending U.S. patent application Ser. No. 09/716,890, which is incorporated herein by reference in entirety. Commercially, PHI-5 is available as the wound dressing material Dermax®. It has been shown that PHI-5 has a positive influence on wound healing, when applied in chronic wounds that were not responding to conventional therapeutical interventions. See, Effect of Polyhydrated Ionogen in Cultures of Normal and Diabetic Human Dermal Fibroblasts, S. Monroe, PhD, E. M. Sampson, MS, M. P. Popp, PhD, R. Lobmann, MD, and G. S. Schultz PhD. Examples of wound-care applications where the PHI technology has been evaluated include chronic diabetic foot ulcers and Stage III decubitus (pressure) ulcers and wounds associated with insertion of a medical implant devices. These types of chronic wounds are problematic on account of the overproduction of matrix matalloproteases (MMP's), zinc-dependent proteins produced in response to tissue damage.

MMP's are a normal part of the body's response to routine local tissue damage and, in the normal response pattern, help to remove damaged tissue from the injured area and prepare the afflicted area for the growth of replacement tissue. However, in chronic wounds, MMP's are overproduced resulting in the breakdown and destruction of newly-regenerated tissue. This abnormal response results in either slow healing of the wound or prevents healing altogether. In the wounds formed during insertion of a medical device, this abnormal response has the potential to negatively interfere with the body's acceptance of the medical implant it results in the wounds continuing to resist healing. The PHI formulation is found to act locally at the injured tissue by affecting matrix metalloprotease (MMP) levels. More specifically, the PHI formulation down regulates MMP levels, which helps to restore the environment in and around the wound and promote a more normal wound-healing response. Thus, the PHI formulation would be advantageous for treating wounds resulting from the insertion of a medical implant, thereby improving the probability of implant acceptance by the surrounding tissue. It is therefore desirable to develop a method for providing continuous delivery of the PHI ions to wounds located at the interface between implant and the surrounding body tissue. However, since PHI-5 is a water-soluble ionic substance, it is probably released even faster than a protein. In fact, clinicians are usually re-applying the PHI-5-containing bandages daily. Thus, there further exists a need for methods of sustained release of the PHI-5 substance to wound sites.

### SUMMARY OF THE INVENTION

Various authors already described an imbalance of matrix metalloproteinases (MMPs), and of MMP inhibitors, in chronic wound tissue and -fluid. It is assumed that PHI-5 has the capability to correct such imbalance between MMPs and MMP-inhibitors. Previously, many studies have shown comparable inhibition of several proteinases by zinc and other divalent metal ions (copper, cadmium, nickel, calcium) from a variety of chemical and organic sources. It has now been found that microtextured silicone wound covers loaded with PHI-5 can improve wound healing, when placed in a standardized full-thickness cutaneous wound in vivo. Through studies of the efficacy of PHI-5 in a standardized animal model, it has been discovered that delivering the PHI-5 composition to the wound site via silicone wound covers significantly improves the initial efficacy of wound healing.

Standardized studies were performed on guinea pigs having identical full thickness cutaneous wounds, wherein the wounds were treated with varying initial concentrations of PHI-5 delivered on a silicone substrate implanted in the wound site. The results showed a significant decrease in wound size in the first week healing corresponding to the concentration of PHI-5 delivered. Subsequent analysis at three and six weeks showed that no significant differences in the wound size between wounds. These initial results were achieved without use of a sustained release formula of PHI-5, thus it is believed that the significant initial increase in efficacy of the wound healing was a result of the initial week long application of the silicone impregnated wound cover. Addition of an extended, slow release carrier to the PHI-5 loaded onto a micro-textured silicone pad or bioabsorbable implant will further improve the efficacy of wound healing. Any sustained release carriers or methods for sustained release known in the art may be used in combination with the PHI-5 to achieve varying periods of release for the PHI-5 ions and thereby improve the efficacy of PHI-5 in treating chronic wounds, including without limitation: combination with microparticles, collagen or the salts of growth factors, encapsulation in biodegradable microsphere formulations, combination with films or sustained release foams.

For example, in an embodiment, PHI-5 may be combined with biodegradable polyester homopolymers, such as polyglycolide, polyactide, and poly(DL-lactide-co-glycolide), before being loaded on the micro-textured silicone pad to further extend the release time period of the PHI-5. Here, the polymers degrade with exposure to aqueous environments, such as biological fluids, until the polymer device loses its mechanical integrity, thereby releasing the micro-encapsulated PHI-5 formulation. Degradation rates of the polymers, and therefore delivery rate of the encapsulated PHI-5 formulation, may be varied with the type of polymer used and specific composition of the polymer.

Alternatively, a collagen delivery system may incorporate the PHI-5 ions into bioabsorbable collagen pads and then as the collagen is biosorbed at the wound site, the ions will be delivered. In an alternative embodiment, the PHI-5 may be loaded directly onto nanospun fibers and collagen pads. In another alternative embodiment, a multilayered system incorporating foams that will slow down the migration of the ions into the implant bed.

In an alternative embodiment, the PHI-5 formulation may be combined using salts of growth factors. Systems for the growth factors themselves have been developed for use with time release systems including PLGA delivery and liposomal delivery. Here, the same system would be used with the salts of growth factors. In an alternate embodiment, the PHI salts may be delivered via liposomal delivery. In this embodiment, the PHI-5 salts may be encapsulated in a non-polar delivery system.

For superficial wounds, it is further believed multiple applications of impregnated silicone pads containing a sustained release formulation of PHI-5 may further improve the efficacy of wound healing. Additionally, for subcutaneous wounds associated with the insertion of a medical implant, for example, a dental implant, the PHI-5 impregnated membranes of the present invention wherein the PHI-5 is contained within a timed release formulation may be particularly advantageous by providing for continuous delivery of the PHI-5 formulation to the wound site over varying time periods. In addition, the PHI-5 impregnated pads may be useful in other applications treatment of stage 1V decubitus ulcers.

### BRIEF DESCRIPTION OF THE DRAWINGS

FIG. 1A. is a photo of the surgical procedure of example 1 showing the surgical area as drawn onto the skin using a pre-fabricated steel mold.

FIG. 1B. is a photo of the surgical procedure of example 1 showing an anaesthetized guinea pig with drawing on right flank.

FIG. 1C. is a photo of the surgical procedure of example 1 showing a circular wound, 2 cm Ø, after application of the silicone membrane.

FIG. 1D. is a photo of the surgical procedure of example 1 showing application of bandage.

FIG. 2A. is a photo of a wound of example 1 with calibration ruler at day 7 of the study

FIG. 2B illustrates the Wound Surface Area (WSA) of a wound from example 1 at day 7.

FIG. 2C illustrates the Reference Surface Area (RSA) of a wound from example 1 at day 7.

FIG. 3. illustrates histomorphometrical measurements on a histological section of a wound from example 1 at 3 weeks after surgery wherein A=length of neo-epithelium B=wound opening, C=width of granulation tissue ED=epidermis, HF=hair follicle, CT=connective tissue, GT=granulation tissue, PC=panniculus carnosus, PA=panniculus adiposus.

FIG. 4. illustrates histomorphometrical measurements on a histological section of a wound from example 1 at 6 weeks after surgery wherein the length of superficial granulation tissue is measured at three levels (A, B, C), as well as the narrowest distance between hair follicles (D).

FIG. 5A. depicts the wound appearance of a wound from example 1 at day 7 where all wounds are still open.

FIGS. 5B and C. depict the wound appearance of a wound from example 1 at day 21 where wounds are either B) nearly or C) fully closed.

FIG. 5D. depicts the wound appearance of a wound from example 1 at day 42, where all wounds are closed.

FIG. 6. illustrates the Mean Wound Surface Area (WSA) for example 1 at one week and three weeks

FIG. 7. Mean Reference Surface Area (RSA) for example 1 at one week, three weeks and six weeks.

FIG. 8A shows a histological section at 3 weeks after surgery with a large epithelial defect is still present

FIG. 8B. shows a higher magnification of a histological section at three weeks after surgery showing that the basal cell layer is deficient over the wound bed area.

FIG. 9A. shows a histological section at three weeks after surgery where the whole surgical wound area is covered by a new layer of epithelium

FIG. 9B. shows a higher magnification of a histological section at three weeks after surgery showing that the new epithelial layer contains an intact layer of basal cells.

FIG. 10A. shows a histological section at six weeks after surgery showing all sections are fully covered with epithelium, but contain varying amounts of granulation tissue.

FIG. 10B. shows a histological section at six weeks after surgery showing all sections are fully covered with epithelium, but contain varying amounts of granulation tissue.

### DETAILED DESCRIPTION OF THE INVENTION

To create a silicone delivery pad for PHI-5 for use in the present invention, Medical-grade silicone rubber, for example, polydimethylsiloxane, NuSil MED-4211, NuSil Technology, CA, USA, may be mixed as prescribed. The mixture may then be cast on a silicon template, containing micro-grooves to obtain a single-sided mixtotextured sheet of silicone. In one embodiment, the template may have a groove depth of 1.0 μm and a ridge- and groove-width of 10.0 μm. After polymerization, substrates of may be cut from the produced sheets to create silicone wound pads. The substrates may be of any shape and size suitable for wound coverage. The substrates are then sterilized and the textured size is hydrophilized. In an embodiment, the substrates are hydrophilized by applying a Radio Frequency Glow Discharge (RFGD; argon, 5 minutes). Finally, the substrates are loaded with aliquots of equal volume of PHI-5 and lyophilized. For example, the substrates may be loaded with 1.25, 5.00, 10.00, 15.00, 20.00 or 25.00 μg of PHI-5. Alternatively the substrates may be loaded with a volume of solution containing up to 1% by weight of the total solution, more preferably up to 5% by weight of the total solution, more preferably up to 10% by weight of the total solution.

The PHI-5 formulation may combined with any of the various long acting sustained release formulations or processes to achieve varying time periods of sustained release of the PHI-5 ions, for example, over a period of six hours, alternatively 12 hours, alternatively 24 hours, alternatively 48 hours, alternatively 72 hours, alternatively one week, alternatively two weeks, alternatively three weeks, alternatively one month, alternatively two months, alternatively three months.

In an embodiment, PHI-5 may be combined with biodegradable polyester homopolymers, such as polyglycolide, polyactide, and poly(DL-lactide-co-glycolide), before being loaded on the micro-textured silicone pad to further extend the release time period of the PHI-5. Here, the polymers degrade with exposure to aqueous environments, such as biological fluids, until the polymer device loses its mechanical integrity, thereby releasing the micro-encapsulated PHI-5 formulation. Degradation rates of the polymers, and therefore delivery rate of the encapsulated PHI-5 formulation, may be varied with the type of polymer used and specific composition of the polymer.

Alternatively, a collagen delivery system may incorporate the PHI-5 ions into bioabsorbable collagen pads and then as the collagen is biosorbed at the wound site, the ions will be delivered. In an alternative embodiment, the PHI-5 may be loaded directly onto nanospun fibers and collagen pads. In another alternative embodiment, a multilayered system incorporating foams that will slow down the migration of the ions into the implant bed.

In an alternative embodiment, the PHI-5 formulation may be combined using salts of growth factors. Systems for the growth factors themselves have been developed for use with time release systems including PLGA delivery and liposomal delivery. Here, the same system would be used with the salts of growth factors. Alternatively, the PHI salts may be delivered via liposomal delivery, encapsulated in a non-polar delivery system.

The PHI-5 loaded membranes may then be implanted into a wound site. The membranes may be sutured or otherwise attached to the wound so that the surface contacting the wound contains the sustained release formulation of PHI-5 and provides for continuous delivery of PHI-5 ions to the wound. Subsequently, the wounds containing the impregnated silicone pad may be covered with a sterile dressing. In an embodiment, a semi-permeable polyurethane dressing may be first be used to cover the silicone membrane, followed by sterile mech gauze and surgical tape to further secure the dressing.

The impregnated membranes continuously deliver PHI-5 to the wound site until the membranes is either removed, absorbed or subsumed by the surrounding body tissue. The membranes may be removed at any suitable interval, after the loaded PHI-5 has been absorbed by the wound. In one embodiment, the membranes were removed after one week, however it is envisioned that the membranes may be left attached to the wound for longer or shorter intervals depending on, for example, the type and depth of the wound, the amount of PHI-5 loaded onto the silicone and the time period for sustained release of the PHI-5. For example, the membranes may remain attached to the wound for one week, alternatively two weeks, alternatively three weeks, alternatively one month, alternatively two months, alternatively three months. In an alternate embodiment, a bioabsorbable membrane is absorbed by the surrounding body tissue.

Additionally, the removed membranes may also be replaced with a new silicone membrane impregnated with an equal, less or greater amount of PHI-5 again depending on such factors as the type and depth of wound, progress in treatment, dosage of PHI-5 applied and interval of replacement. For example, the membranes may be removed and replaced monthly, bi-monthly, weekly, bi-weekly or at a shorter interval. The interval of replacement will depend upon factors such as the amount of PHI loaded onto the membrane, the pattern of micro-texture on the membrane, the delivery rate of the timed release formulation, the wound size and the wound location. Since PHI-5 is a water-soluble ionic substance, it is probably released even faster than a protein. Thus, the addition of a long acting sustained release carrier to the PHI-5 and the multiple replacements of the loaded silicone membranes enable a continuous and sustained release of PHI-5 ions to the wound site and thereby improve the efficacy of the PHI-5 in wound healing. The application will now be further described by way of the following, non-limiting examples.

### EXAMPLE 1

A study was performed on the efficacy of PHI-5 in an immediate release formulation to treat full thickness cutaneous wounds in a standardized animal model. Pre-operatively, the animals were shaved thoroughly. Circular full-thickness cutaneous wounds extending to the panniculus carnosus were created on the right flank of each guinea pig, using aseptical techniques. Then, micro-grooved silicone rubber membranes were sutured onto the wound, containing 0 (controls), 1.25, 5.00, or 10.00 μg PHI-5 in an immediate release formulation. The silicone substrates implanted with the side containing PHI-5 making contact with the wound to simulate insertion of a PHI-5 impregnated membrane with a medical implant. The procedures and results of the study are explained in the following paragraphs:

Substrates with PHI-5

Medical-grade silicone rubber (polydimethylsiloxane, NuSil MED-4211, NuSil Technology, CA, USA) was mixed as prescribed. To obtain a single-sided microtexture, the mixture was cast on a silicon template, containing micro-grooves with a groove depth of 1.0 μm and a ridge- and groove-width of 10.0 μm (C2V, Enschede, the Netherlands). After polymerization, coin-shaped substrates of 20 mm diameter were cut from the produced sheets. Substrates were washed in 10% liquinox solution (Alconox, New York, N.Y., USA), cleaned ultrasonically, and rinsed thoroughly in reverse osmosis water (MilliQ, Millipore Corp, Bedford, Mass., USA). Subsequently, they were washed in 70% and 100% ethanol and dried to air. The membranes were autoclaved for sterilisation at 121° C. for 15 minutes. A Radio Frequency Glow Discharge (RFGD; argon, 5 minutes) treatment was applied to remove surface fouling, and to hydrophilize the textured side. Finally, the membranes were loaded with aliquots of equal volume, containing 0, (controls), 1.25, 5.00 and 10.00 μg of PHI-5, and lyophilized overnight.

Application Procedure

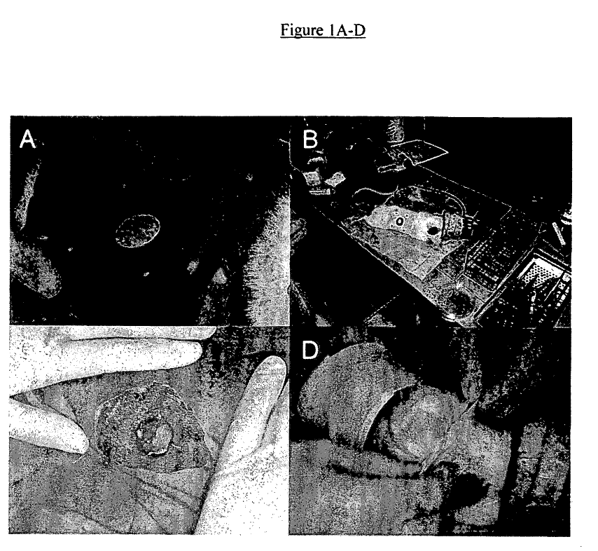

Pre-operatively, the animals were shaved thoroughly. Surgery was performed under general inhalation anesthesia of O2, N2O, and isoflurane. Prior to creating the wound, local anesthesia was given by infiltration with lidocain 2% including adrenalin. The skin was scrubbed with iodine, and subsequently, standardized orientation points to measure wound contraction were created with tattooing ink, using fixed holes in a pre-made steel mold. As shown in FIG. 1A, the center of this mold contained a 20 mm □ circular hole, used to mark the amount of tissue to be excised. Then, the circular full-thickness cutaneous wounds extending to the panniculus camosus were created on the right flank of each guinea pig, using aseptical techniques. As shown in FIG. 1C, the silicone substrates were sutured onto the wound, with the side containing PHI-5 making contact with the wound. Subsequently, wounds were covered with semi-permeable polyurethane dressings (Tegaderm, 3M Co, Minneapolis, Mn, USA). One layer of dry sterile fine mesh gauze (Tendra Mesoft 5×5 cm, Mölnlycke, Göteborg, Sweden) was applied onto the Tegaderm, and the dressings were secured in place with two circular layers of surgical tape (Elastoplast-E 6 cm, Beiersdorf, Spain). Special attention was paid to the design of the bandage. The outer layer of tape was also wrapped in front of the fore legs, thus preventing the guinea pigs to remove it by paw movement or chewing (FIG. 1D). After one week, the PHI-5 containing silicone membranes were removed, after which the bandages were reapplied. After 3 or 6 weeks, the wound and all surrounding tissues were retrieved for histological and histomorphometrical analyses.

Morphometrical Evaluation of the Wounds

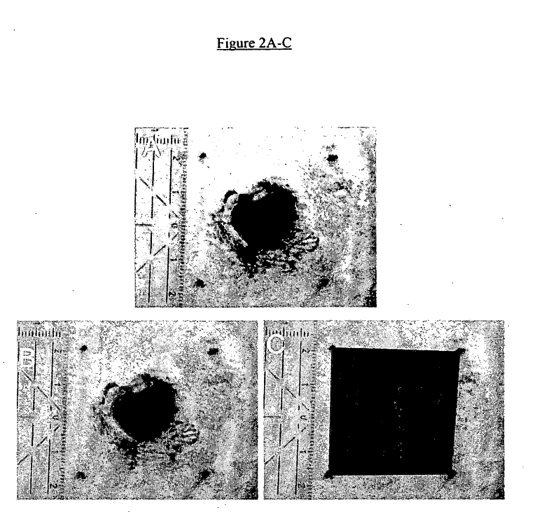

Standardized digital wound photographs were taken on days 7, 21 and 42, using a digital camera on macro setting. Photographs were calibrated to distance with a ruler on each photograph, using Leica Qwin software (Leica Microsystems Imaging Solutions, Ltd, UK). Per photograph, two measurements were made, see FIGS. 2A-C, of Wound Surface Area (WSA) and Reference Surface Area (RSA). These indicate the amount of wound closure, and the amount of wound contraction respectively.

Histological Evaluation Techniques

After retrieval, the excised tissue was fixed in 4% buffered formaldehyde for twenty-four hours, dehydrated in a series of ethanol, and embedded in paraffin. Thereafter, 6 μm sections were cut using a Leica RM 2165 Microtome. Every 25th section was collected and stained with haematoxilin and eosin (Merck, Darmstadt, Germany).

Histomorphometry

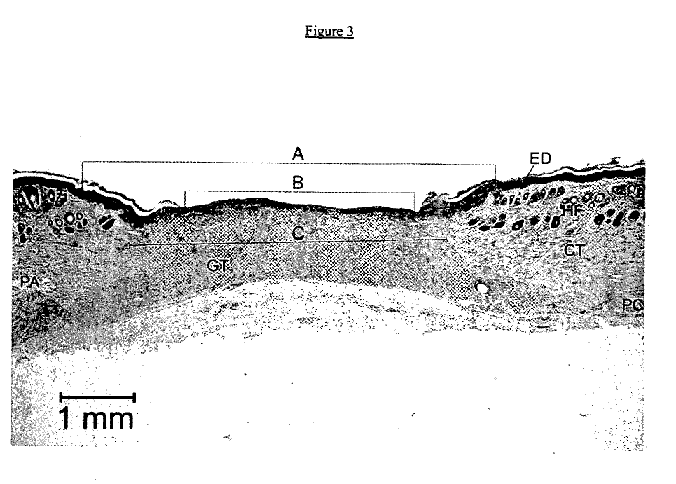

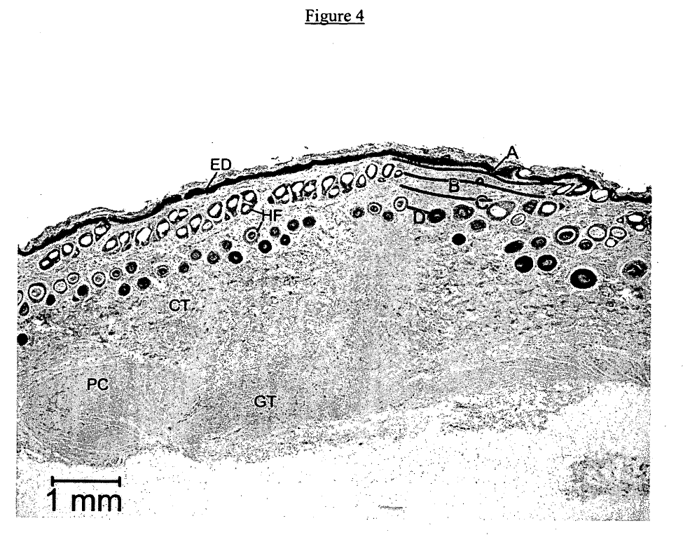

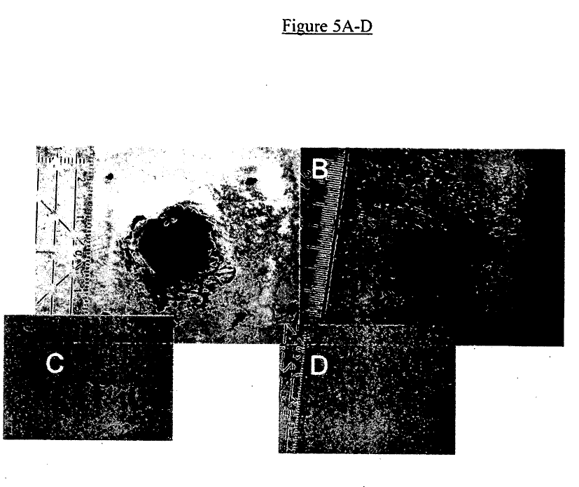

Computer-based image analysis of re-epithelialization, wound area, and granulation tissue was performed on histological images. Per wound three histology sections, 150 μm apart, were selected for evaluation. In the three-week sections, three parameters were measured at pre-determined locations in the wound: length of the neo-epithelial layer, size of wound opening, and narrowest width of the granulation core See FIG. 3. In the six-week sections, measurements were made in three layers of the superficial granulation tissue, thereafter calculating the mean length of the superficial granulation tissue. Also, the narrowest distance between hair follicles (representing the edges of the original skin tissue) on either side of the originally excised wound tissue, was measured See FIG. 4

Statistical Analysis

The averages and standard deviations of data from the quantitative measurements were calculated. Then, data were compared with a one-way ANOVA and a Tukey post-hoc test, using InStat software (v3.05, GraphPad InStat software, GraphPad Inc.). A p value below 0.05 was considered to be significant.

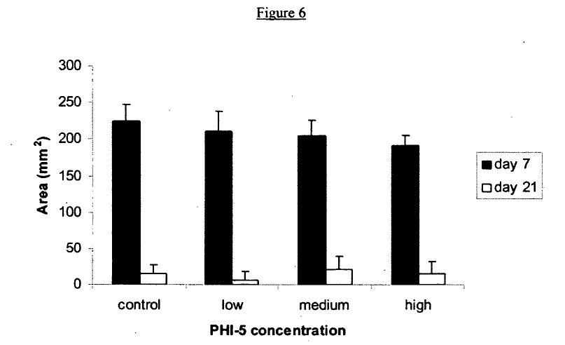

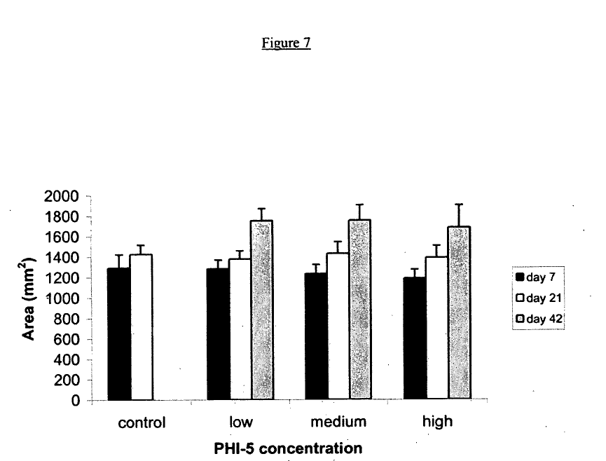

FIGS. 2A-C and 5 A-D show the measurements performed on standardized digital wound photographs after 1, 3, and 6 weeks. Also, wound tissue was excised after 3 and 6 weeks for histological and histomorphometrical evaluation as shown in FIGS. 3, 4, 8A-B, 9A-B, and 10A-B. Results showed a faster wound closure after one week when an increasing concentration of PHI-5 was applied. Specifically, as shown in FIG. 6, after one week the wound photographs showed a decrease in Wound Surface Area (WSA) for the higher PHI-5 concentrations. Especially, a significant result was found between the control group and the highest concentration group. After three and six weeks however, no differences among study groups were found in any of our measurements. These results were achieved using an immediate release formulation of the PHI-5 composition. One plausible explanation for finding only short-term effects lies in the release pattern of PHI-5. From an earlier study we already know that proteinaceous growth factors are released from microtextured surfaces, within 24 hrs in a burst-like manner. Since PHI-5 is a water-soluble ionic substance, it is probably released even faster than a protein therefore the initial application was absorbed within 1-2 days of delivery. The significant improvement in week one reflects the initial treatment with the immediate release formulation. However, after three weeks without continued application of the PHI-5 ions, no significant differences were found among the experimental groups for the length of the wound opening, neo-epithelium, or granulation tissue (Table 1). Also, in the six-week groups without continued application of the PHI-5 ions, no significant differences in length of the superficial granulation tissue or distance between hair follicles were found (Table 2).

Histology

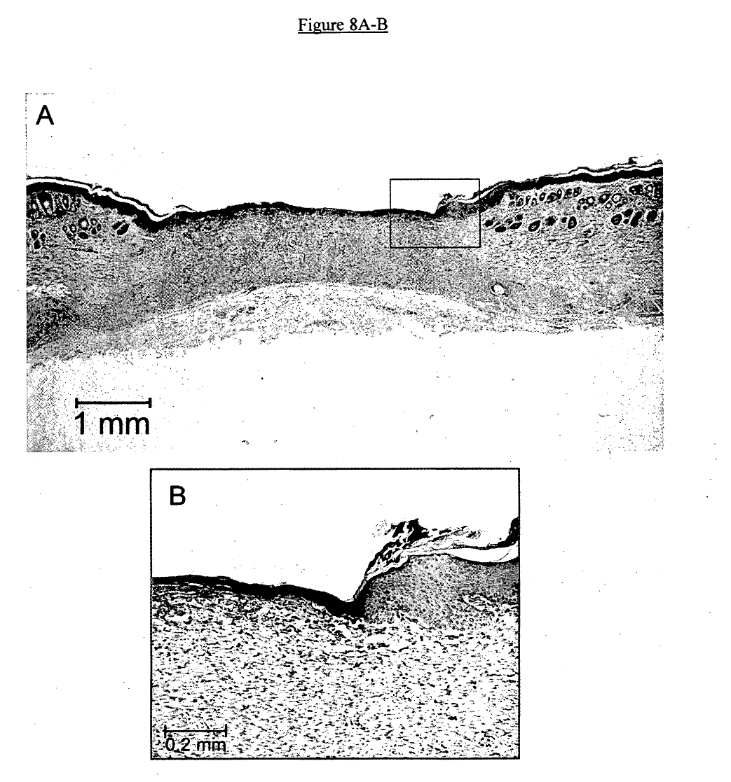

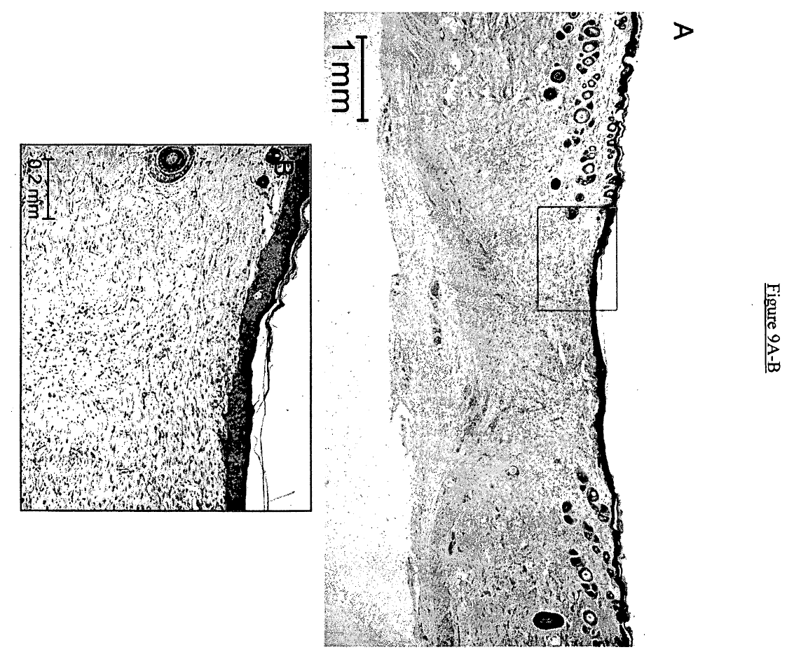

Three weeks after surgery, the excised skin was replaced by a varying amount of granulation tissue, consisting of fibrinoid material and inflammatory cells (FIG. 8A-B, 9A-B). Of all excised wounds, two seemed to still have an intact panniculus carnosus. Re-epithelialisation was observed over the woundbed area, although only five wounds (all in the control and the low concentration groups) were fully covered by an intact epithelium, containing a recognisable basal cell layer. When a defect of epithelial lining was still present, many superficial capillaries were seen, as well as thickening of the epithelium at the wound edges.

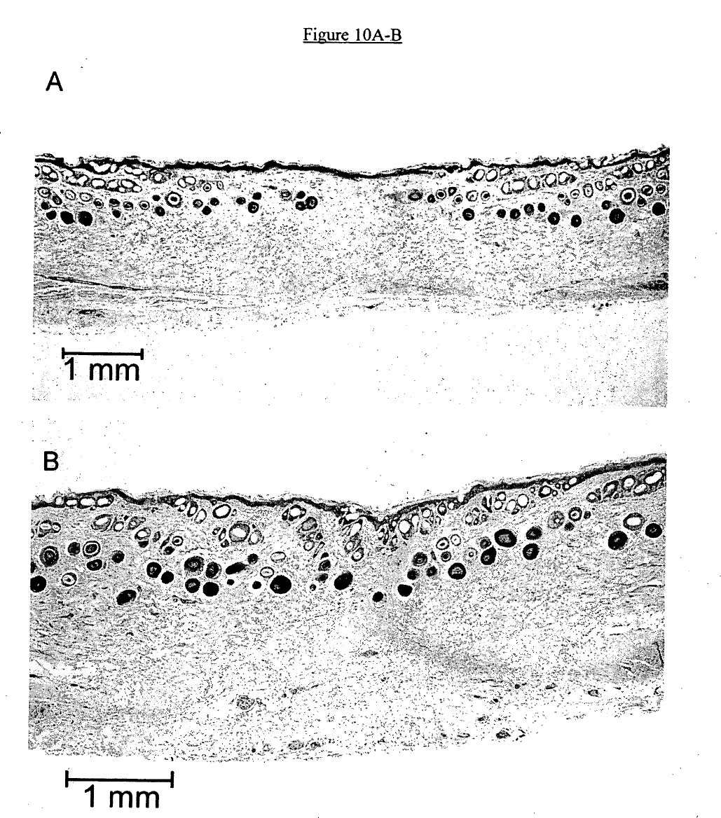

In all sections of the 6-week specimens, an intact keratinizing squamous epithelial lining was seen, which in some cases showed the start of rete peg formation (FIG. 10). Just below the epithelium, a varying amount of granulation tissue was present, becoming narrower at the level of the hair follicles and connective tissue. Even deeper in the sections, a broad area of granulation tissue was always predominantly present, in between both sides of the pre-existent panniculus carnosus.

Histomorphometry

Table 1 shows the average histomorphometrical measurements and standard deviations after 3 weeks (mm). No significant differences between different concentration groups were found in width of epithelial defect, granulation core width, or length of neo-epithelium.

TABLE 1

| | Control (SD) | Low (SD) | Medium (SD) | High (SD) |
|---|---|---|---|---|
| Epithelial | 1.20 (1.64) | 1.17 (1.61) | 2.29 (1.96) | 2.99 (1.09) |
| defect | | | | |
| Granulation | 2.48 (1.02) | 3.18 (1.51) | 2.85 (0.72) | 2.60 (1.09) |
| core width | | | | |
| Neo-epi- | 4.94 (2.06) | 5.48 (2.25) | 5.99 (1.68) | 5.51 (1.72) |
| thelial | | | | |
| length | | | | |

Table 2 shows the average histomorphometrical measurements and standard deviations after 6 weeks (mm). No significant differences between different concentration groups were found in width of superficial granulation tissue or in narrowest follicle distance among groups.

TABLE 2

| | Low (SD) | Medium (SD) | High (SD) |
|---|---|---|---|
| Granulation width | 1.57 (0.46) | 0.84 (0.66) | 1.19 (0.54) |
| Follicle distance | 1.35 (0.87) | 0.59 (0.54) | 0.60 (0.46) |

Like with the animal model, a specific annotation has to be made on the use of our delivery vehicle of PHI-5 into the wound bed area. It might be suggested, that suturing a silicone membrane onto the wound could have the effect of splinting the wound open, and prevent wound contraction in the first week. However, in a previous study it was already proven that there are no differences between the control group, wearing a silicone membrane, and a sham group, having the wound left open.

Considering our measurements, after one week the wound photographs showed a decrease in Wound Surface Area, for the higher PHI-5 concentrations. Especially, a significant result was found between the control group and the highest concentration group. This means we can, at least partially, maintain our hypothesis that microtextured polymeric membranes loaded with PHI-5 can improve wound healing, when placed in a full-thickness cutaneous wound in vivo. However, two weeks later, no significant differences between groups could be measured anymore. One plausible explanation for finding only short-term effects lies in the release pattern of PHI-5. From an earlier study we already know that proteinaceous growth factors are released from microtextured surfaces, within 24 hrs in a burst-like manner 29. Since PHI-5 is a water-soluble ionic substance, it is probably released even faster than a protein. In fact, clinicians are usually re-applying the PHI-5-containing bandages daily. Thus, follow-up studies in the animal model should be directed to multiple deliveries, or involve an appropriate slow-release carrier. Next to the time frame of delivery, the efficacy of PHI-5 could also be dependent on the dose. The greatest effect of PHI-5 was measured, when applied in our high concentration of 10.00 μg per wound. Even higher concentrations would have to be tested, to find the optimum level for treatment.

### EXAMPLE 2

A long acting time release formulation of PHI-5 is prepared using a biodegradable polymer to microencapsulate the PHI-5 ions. The aliquots of the long acting dosage formulation containing 1.25, 5.00, 10.00, 15.00, 20.00 and 25.00 μg of the PHI-5 composition are loaded onto microtextured silicon membranes. The wound is cleaned with rubbing alcohol to remove any contamination and the silicone substrates were sutured onto the wound, with the side containing PHI-5 making contact with the wound. Subsequently, wounds were covered with semi-permeable polyurethane dressings and the PHI-5 loaded silicone membranes are left on the wound for one week, two weeks and one month. The results with the sustained release formulation show significant improvements in wound healing.

### EXAMPLE 3

A long acting time release formulation of PHI-5 is prepared using a collagen delivery system. Then aliquots of the long acting dosage formulation containing 1.25, 5.00, 10.00, 15.00, 20.00 and 25.00 μg of the PHI-5 composition are loaded onto microtextured silicon membranes. The wound is cleaned with rubbing alcohol to remove any contamination and the silicone substrates were sutured onto the wound, with the side containing PHI-5 making contact with the wound. Subsequently, wounds were covered with semi-permeable polyurethane dressings which are left on the wound for one week. After one week, the silicone membranes are removed and replaced with new silicone membranes loaded with the same dosage of the PHI-5 formulation in the collagen delivery system. The second silicone membrane is sutured onto the wound site and covered with semi-permeable polyurethane dressings which are left on the wound for one week. The results with multiple uninterrupted applications of the sustained release formulation show significant improvements in wound healing.

What is claimed is:

1. A method for treating wounds associated with the insertion of a medical implant comprising the steps of:
applying an effective amount of a time release formulation of a therapeutic composition comprising therapeutically effective amounts of potassium ions, calcium ions and zinc ions to a surface of a silicone membrane, wherein time release formulation provides for delivery of the therapeutic composition over a period of time; and
implanting the membrane in a wound site so that the side containing the therapeutic composition contacts the wound.

2. The method of claim 1, wherein the membrane is a silicone membrane.

3. The method of claim 1, wherein the membrane is a bioabsorbable membrane.

4. The method of claim 3, wherein the bioabsorbable membrane is a collagen membrane.

5. The method of claim 1, wherein the membrane is implanted subcutaneously.

6. The method of claim 1, wherein the time release formulation provides for sustained delivery of the therapeutic composition over a period of 12 hours.

7. The method of claim 1, wherein the time release formulation provides for sustained delivery of the therapeutic composition over a period of 24 hours.

8. The method of claim 1, wherein the time release formulation provides for sustained delivery of the therapeutic composition over a period of 48 hours.

9. The method of claim 1, wherein the time release formulation provides for sustained delivery of the therapeutic composition over a period of 72.

10. The method of claim 1, wherein the time release formulation provides for sustained delivery of the therapeutic composition over a period of one week

11. The method of claim 1, wherein the time release formulation provides for sustained delivery of the therapeutic composition over a period of two weeks.

12. The method of claim 1, wherein the time release formulation provides for sustained delivery of the therapeutic composition over a period of three weeks.

13. The method of claim 1, wherein the time release formulation provides for sustained delivery of the therapeutic composition over a period of one month.

14. The method of claim 2, wherein a surface of the silicon membrane is microtextured and wherein the therapeutic composition is applied to the microtextured surface.

15. The method of claim 2, wherein the micro-texture comprises a plurality of micro-grooves running longitudinally across the surface of the silicone membrane.

16. The method of claim 15, wherein the micro-grooves have a depth of at least 1.0 μm.

17. The method of claim 15, wherein the micro-grooves have a groove width of at least 10.0 μm.

18. The method of claim 1, wherein therapeutic composition is PHI-5.

19. The method of claim 18, wherein a daily dosage corresponding to 1.25 μg of the therapeutic composition is loaded onto the silicone membrane.

20. The method of claim 18, wherein a daily dosage corresponding to 5.0 μg of the therapeutic composition is loaded onto the silicone membrane.

21. The method of claim 18, wherein a daily dosage corresponding to 10.0 μg of the therapeutic composition is loaded onto the silicone membrane.

22. The method of claim 18, wherein a daily dosage corresponding to 15.0 μg of the therapeutic composition is loaded onto the silicone membrane.

23. The method of claim 18, wherein a daily dosage corresponding to 20.0 μg of the therapeutic composition is loaded onto the silicone membrane.

24. The method of claim 18, a volume of solution containing up to 0.25% by weight of the therapeutic composition is loaded onto the silicone membrane.

25. The method of claim 18, a volume of solution containing up to 1% by weight of the therapeutic composition is loaded onto the silicone membrane.

26. The method of claim 18, a volume of solution containing up to 5% by weight of the therapeutic composition is loaded onto the silicone membrane.

27. The method of claim 18, a volume of solution containing up to 10% by weight of the therapeutic composition is loaded onto the silicone membrane.

28. The method of claim 1, further comprising the steps of:
removing the membrane from the wound site; and
loading a second membrane with an effective amount of the therapeutic composition; and
applying the second membrane to the wound, so that the side containing the therapeutic agent contacts the wound.

29. The method of claim 28, wherein the amount of the therapeutic agent applied to the second membrane is the same as the amount applied to the first silicone membrane.

30. The method of claim 28, wherein the amount of the therapeutic agent applied to the second membrane is greater than the amount applied to the first silicon membrane.

31. The method of claim 28, wherein the amount of the therapeutic agent applied to the second membrane is less than the amount applied to the first silicone membrane.

32. The method of claim 28, wherein the membrane is replace weekly.

33. The method of claim 28, wherein the membrane is replaced bi-weekly.

34. The method of claim 28, wherein the membrane is replaced monthly.

35. The method of claim 28, wherein the membrane is replaced bimonthly.

36. The method of claim 28, further comprising the steps of removing the silicone membrane from the wound and applying a new membrane loaded with an effective amount of the therapeutic agent multiple times.

37. The method of claim 1, wherein the wound is a full thickness cutaneous wound.

38. The method of claim 1, wherein the wound is a decubitus ulcer.

39. A method for treating wounds resulting from a medical implant comprising the step of applying an effective amount of a time release formulation of a therapeutic composition comprising therapeutically effective amounts of potassium ions, calcium ions and zinc ions to a surface of the medical implant before the dental implant is implanted in a patients jaw, wherein time release formulation provides for delivery of the therapeutic composition to the implant site over a period of time.

40. The method of claim 39, wherein therapeutic composition is PHI-5.

41. The method of claim 40, wherein the time release formulation provides for sustained delivery of the therapeutic composition over a period of one week.

42. The method of claim 40, wherein the time release formulation provides for sustained delivery of the therapeutic composition over a period of two weeks.

43. The method of claim 40, wherein the time release formulation provides for sustained delivery of the therapeutic composition over a period of three weeks.

44. The method of claim 40, wherein the time release formulation provides for sustained delivery of the therapeutic composition over a period of one month.

45. The method of claim 40, wherein the time release formulation provides for sustained delivery of the therapeutic composition over a period of two months.

46. The method of claim 40, wherein the time release formulation provides for sustained delivery of the therapeutic composition over a period of three months.

47. The method of claim 40, wherein the time release formulation provides for sustained delivery of the therapeutic composition over a period of three months.
